(19) United States
(12) Patent Application Publication
(10) Pub. No.: US 2026/0012334 A1
(43) Pub. Date: Jan. 8, 2026

(54) SECURE CONFIGURATION OF MEDICAL DEVICES

(71) Applicant: ICU Medical, Inc., San Clemente, CA (US)

(72) Inventors: Hrishikesh Anil Dandekar, Pune (IN); Mark C. Rohlwing, Mesa, AZ (US); Satyendra Singh Jadaun, Kota (IN); Dharani Kumar Srinivasan, Chennai (IN); Rahul K R, Chennai (IN)

(21) Appl. No.: 19/210,938

(22) Filed: May 16, 2025

Related U.S. Application Data
(63) Continuation of application No. PCT/US2024/058292, filed on Dec. 3, 2024.
(60) Provisional application No. 63/649,037, filed on May 17, 2024.

(30) Foreign Application Priority Data
Dec. 5, 2023 (IN) 202311082782

(51) Int. Cl. H04L 9/08; H04L 9/32

(52) U.S. Cl. CPC H04L 9/0825; H04L 9/321; H04L 9/3268

(57) ABSTRACT

The present disclosure is directed to secure configuration of network-connected electronic medical devices, in some cases regardless of whether the devices are in a clinical environment, factory, or other location.

### CROSS-REFERENCE TO RELATED APPLICATIONS

This application is a continuation application of PCT Application No. PCT/US2024/058292, filed Dec. 3, 2024, and titled “SECURE CONFIGURATION OF MEDICAL DEVICES,” which claims priority to U.S. Provisional Patent Application No. 63/649,037, filed on May 17, 2024 and titled “SECURE MEDICAL DEVICE BOOTSTRAPPING,” and to India Provisional Patent Application No. 20/231,1082782, filed Dec. 5, 2023 and titled “SECURE BOOTSTRAPPING OF INFUSION PUMP USING MOBILE APP,” the contents of each of which are incorporated by reference herein and made part of this specification.

### TECHNICAL FIELD

This disclosure relates to the field of medical device management, and particularly to systems and methods for secure configuration of medical devices.

### BACKGROUND

Electronic medical devices often have processors and other computing components. Such medical devices may execute software and communicate with other computing systems via a network. Secure network communication may involve establishing secure connections based on the identities of the devices participating in the communication, and encryption and decryption of data transmitted via the secure connections.

### SUMMARY OF SOME EMBODIMENTS

In some aspects, the techniques described herein relate to a system for secure configuration of medical devices, the system including: a medical device including one or more processors, a secure memory, and a first wireless network interface; and a setup device including one or more processors and a second wireless network interface; wherein the medical device is configured to: present encoded device data representing a public key of the medical device, a device identifier of the medical device, and a session identifier of a direct communication session to be established with the setup device; receive, from the setup device, direct connection data including credentials, encrypted using the public key, for establishing the direct communication session; decrypt the credentials using a private key corresponding to the public key; establish the direct communication session using the credentials; and receive, from the setup device via the direct communication session, configuration data for communicating with a server over a clinical network; and wherein the setup device is configured to: scan the encoded device data presented by the medical device; generate the direct connection data, including the credentials, encrypted using the public key; broadcast the direct connection data; establish the direct communication session with the medical device; and send the configuration data to the medical device.

In some aspects, the techniques described herein relate to a computer-implemented method for secure configuration of medical devices, including: as performed by a medical device including one or more processors, a secure memory, and a first wireless network interface, presenting encoded device data representing a public key of the medical device, a device identifier of the medical device, and a session identifier of a direct communication session to be established with a setup device; receiving, from the setup device, direct connection data including credentials, encrypted using the public key, for establishing the direct communication session; decrypting the credentials using a private key corresponding to the public key; establishing the direct communication session using the credentials; and receiving, from the setup device via the direct communication session, configuration data for communicating with a server over a clinical network.

In some aspects, the techniques described herein relate to an infusion pump including one or more processors, a secure memory, and a first wireless network interface, wherein the infusion pump is configured to: present encoded device data representing a public key of the infusion pump, a device identifier of the infusion pump, and a session identifier of a direct communication session to be established with a setup device; receive, from the setup device, direct connection data including credentials, encrypted using the public key, for establishing the direct communication session; decrypt the credentials using a private key corresponding to the public key; establish the direct communication session using the credentials; and receive, from the setup device via the direct communication session, configuration data for communicating with a server over a clinical network.

In some aspects, the techniques described herein relate to a non-transitory computer-readable storage medium storing computer-executable instructions, wherein the computer-executable instructions configure a medical device to at least: present encoded device data representing a public key of the medical device, a device identifier of the medical device, and a session identifier of a direct communication session to be established with a setup device; receive, from the setup device, direct connection data including credentials, encrypted using the public key, for establishing the direct communication session; decrypt the credentials using a private key corresponding to the public key; establish the direct communication session using the credentials; and receive, from the setup device via the direct communication session, configuration data for communicating with a server over a clinical network.

### BRIEF DESCRIPTION OF THE DRAWINGS

Embodiments of various inventive features will now be described with reference to the following drawings. Throughout the drawings, reference numbers may be re-used to indicate correspondence between referenced elements. The drawings are provided to illustrate example embodiments described herein and are not intended to limit the scope of the disclosure.

FIG. 1 is a block diagram of an example network environment including a medical device, setup device, and server according to some embodiments.

FIG. 2 is a diagram illustrating data flows and interactions during a medical device setup procedure according to some embodiments.

FIG. 3 is a block diagram illustrating data flows and interactions during a medical device setup procedure according to some embodiments.

FIG. 4 is a block diagram illustrating data flows and interactions for applying a current timestamp to a time setting of a medical device during a medical device setup procedure according to some embodiments.

FIG. 5 is a flow diagram of an illustrative routine performed by a medical device to process a current timestamp received in connection with a medical device setup procedure according to some embodiments.

### DETAILED DESCRIPTION OF ILLUSTRATIVE EMBODIMENTS

The present disclosure is directed to secure setup and configuration of network-connected electronic medical devices, in some cases regardless of whether the devices are in a clinical environment, factory, or other location.

Setting up a network-connected electronic medical device—referred to herein simply as a “medical device” for brevity—typically involves providing network configuration information that the medical device will use to communicate over one or more networks with servers, other medical devices, or other systems. For example, configuration information such as network name, address, credentials, and other information may be provided to a medical device to setup the medical device for operation in a clinical network environment. Once a medical device is configured to access a network, the medical device may update installed software, obtain databases (e.g., drug libraries), receive operating instructions, and the like.

Some methods of medical device setup involve manual entry of configuration information into the medical device. For example, an administrator or field engineer may access a user interface of the medical device and enter configuration information such as network name and access credentials for connecting to a network in a clinical environment. While such manual entry may not be burdensome for configuring a single medical device, it can be impractical to manually configure dozens, hundreds, or more medical devices being deployed to a clinical environment. Automated methods of configuring medical devices present their own challenges. For example, if at the time of manufacture the medical devices are configured with default network connectivity information (e.g., addresses, credentials), then those medical devices may be at risk of attacks targeted at the default configuration.

Some aspects of the present disclosure address the issues noted above, among others, through the use of a setup device configured to obtain medical device-specific information from a medical device that is to be setup. The setup device uses the medical device-specific information to establish a secure wireless connection with the medical device and perform a setup procedure by which the medical device may be configured automatically or otherwise without substantial manual user input. Thus, the medical device is setup securely and accurately, without the need for default network connectivity information or sensitive information hardcoded/embedded on the medical device, and without manual entry of connectivity information to the medical device. Moreover, the substantially automatic nature of the setup allows a single administrator or field engineer to setup multiple such medical devices (dozens, hundreds, or more) in a fraction of the time. In some embodiments, the same setup device can configure individual medical devices different from each other (e.g., based on the make, model, or desired use of the medical devices), or apply different configurations to individual devices of a set of devices (whether the same or different devices).

Various aspects of the disclosure will now be described with regard to certain examples and embodiments, which are intended to illustrate but not limit the disclosure. Although aspects of some embodiments described in the disclosure will focus, for the purpose of illustration, on particular examples of medical devices, secure connection types, and the like, the examples are illustrative only and are not intended to be limiting. In some embodiments, the systems and methods described herein may be applied to additional or alternative medical devices, secure connection types, etc. Any feature used in any embodiment described herein may be used in any combination with any other feature, without limitation.

### Overview of Example Network Environment

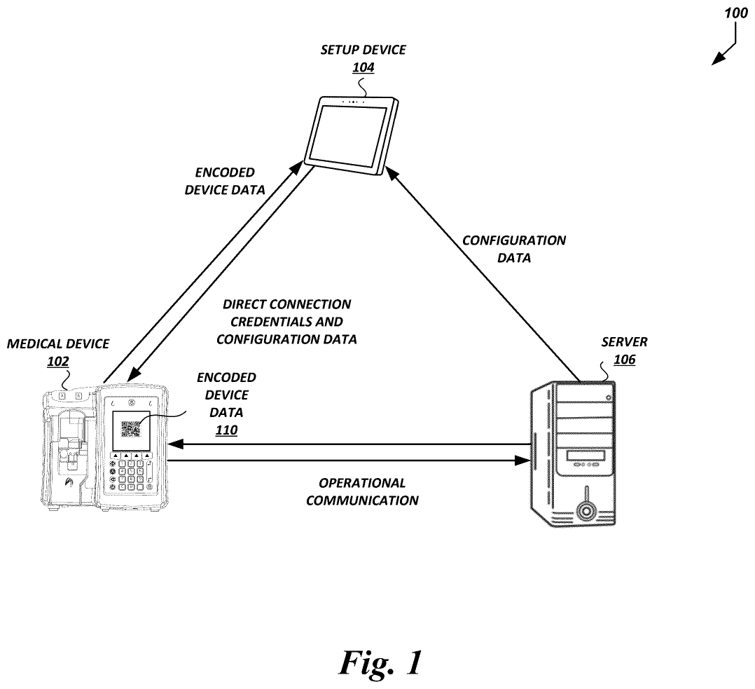

FIG. 1 shows a network environment 100 in which aspects of the present disclosure may be implemented for secure configuration of medical devices.

The network environment 100 may include any number of medical devices 102, setup devices 104, servers 106, other systems, or any combination thereof. The network environment 100 may include one or more wired and/or wireless networks, such as local area networks (“LANs”), virtual local area networks (“VLANs”), wide area networks (“WANs”), the internet, etc. Individual devices or systems may communicate with each other via the one or more wired and/or wireless networks.

The network environment 100 may be implemented within—or may include—one or more clinical facilities (e.g., hospitals or other healthcare facilities), each of which may include various devices and/or systems. In some embodiments, the network environment 100 may be implemented within—or may include—a medical device manufacturing facility, a medical device wholesaler or retailer facility, or the like. Aspects of the present disclosure may be used in or across any such environments.

A medical device 102 may be any electronic medical device, or medical device with an electronic component, configured to communicate with other devices over a communication network. In some embodiments, as shown, a medical device 102 may be an infusion pump.

The setup device 104 may be any electronic device configured to electronically communicate with other devices. In some embodiments, the setup device 104 may be any handheld or substantially portable electronic device with a processor, memory, communication interface, and user interface. For example, the setup device 104 may be a smart phone, personal digital assistant, tablet, laptop computer, desktop computer with a mobile base, etc. The setup device 104 may allow user interaction to initiate setup processes in which the setup device 104 obtains encoded medical device-specific information from a medical device 102 and uses the medical device-specific information to establish a secure connection with the medical device 102. The setup device 104 may then configure the medical device 102, for example to communicate with a server 106, as described in greater detail below.

The server 106 may be any computing system configured to communicate with medical devices 102. For example, the server 106 may be a desktop computer, server computer, network appliance, or the like. In some embodiments, the server 106 may be configured to manage use of the medical device 102. For example, the server 106 may be an on-site server (e.g., within a same clinical environment as the medical device 102) or cloud-based server (e.g., a server physically located in a remote data center accessible via a WLAN or the internet) that manages use of multiple medical devices 102 in or across one or more clinical environments (e.g., by providing drug library information, medication infusion commands, tracking the infusion of medication, etc.). In some embodiments, the server 106 can serve as a gateway for some or all devices and systems of a clinical environment to communicate with a cloud environment. For example, the server 106 may send requests or other communications on behalf of medical devices 102 to another server or servers in the cloud environment, send responses, commands, and other communications from the server(s) in the cloud environment to the medical device, etc.

In some embodiments, the network environment 100 may also or alternatively include one or more clinical information technology systems (not shown). For example, the clinical environment may include a hospital information system (“HIS”) designed to manage the facilities' operation, such as medical, administrative, financial, and legal issues and the corresponding processing of services. The HIS can include one or more electronic medical record (“EMR”) or electronic health record (“EHR”) systems.

The example devices and systems of the network environment 100 shown in FIG. 1 and described herein are illustrative only, and are not intended to be limiting, required, or exhaustive. In some embodiments, a network environment 100 may include additional, alternative, and/or fewer devices and/or systems. For example, although only one instance of a medical device 102, a setup device 104, and a server 106 are shown in FIG. 1, in practice any number or combination of devices and systems may be included in a network environment 100. A single network environment 100 may have dozens, hundreds, or more individual medical devices 102, and the medical devices 102 may be the same as or different than each other.

### Secure Configuration

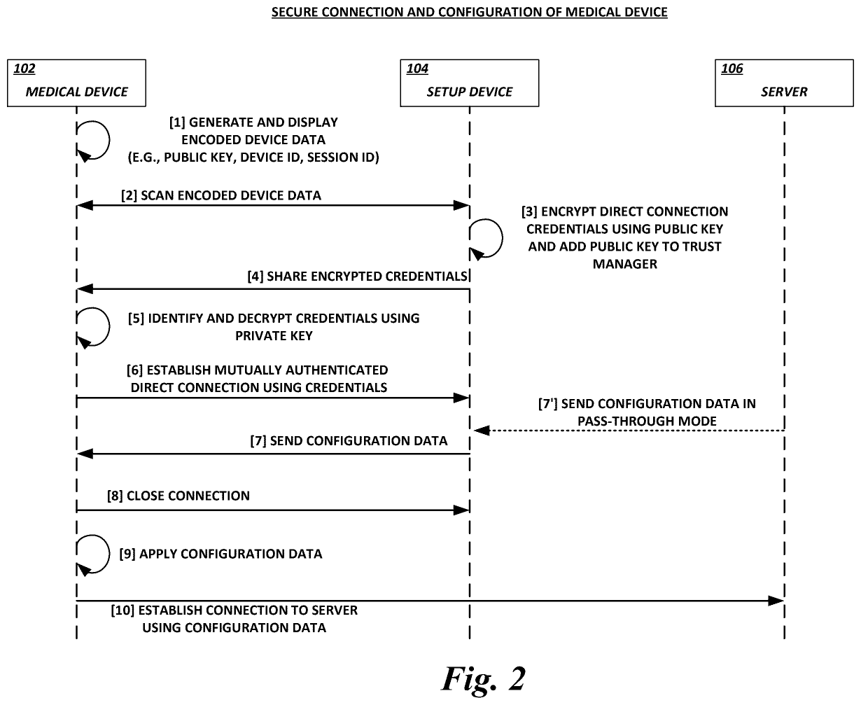

FIG. 2 illustrates example interactions between a medical device 102, setup device 104, and server 106 during the process of configuring the medical device 102 according to some embodiments.

At [1], the medical device 102 may generate and present encoded device data for use by the setup device 104 to establish a secure connection to the medical device 102. By presenting the encoded device data (e.g., on a display), the setup device 104 will be able to scan the encoded device data (e.g. using an optical sensor such as a camera) to obtain the device data without manual entry, and without first establishing a network connection with the medical device 102.

In some embodiments, the device data that the medical device 102 encodes may include a public key that is part of a public-private key pair generated or otherwise maintained by the medical device 102. The corresponding private key—also referred to as the secret key—can be stored in a secure memory area of the medical device 102 from where it can be accessed only for internal cryptographic operations and that is otherwise not accessible outside of the medical device 102. Thus, because the secret key is generated within the medical device 102 and stored in a secure memory area of the medical device 102, the secret key is not exposed to potential security risks involved with generation of the secret key outside of the device (e.g., by a key server), transmission of the secret key over a network (e.g., from the key server), etc. Providing the public key to the setup device 104 allows the setup device 104 to encrypt data that can only be decrypted using the corresponding secret key maintained by the medical device 102. To further increase security, in some embodiments the public-private key pair is single-use. For example, the public-private key pair is discarded and freshly generated every time a setup/configuration process is attempted, making the configuration process more secure than existing methods that use static keys that are never (or less frequently) changed.

In some embodiments, the device data that the medical device 102 encodes may include one or more identifiers, such as a device identifier of the medical device 102, and a session identifier for the communication session to be established between the medical device 102 and setup device 104. Providing such identifiers to the setup device 104 allows the setup device 104 to broadcast an invitation to connect that will be recognized by the medical device 102. In some embodiments, providing such identifiers also lets multiple setup devices 104 to function at the same time for efficiency. Through the identifier in the broadcast, a medical device 102 will be able to find out which one of the broadcasts is meant for itself, among a number of other broadcasts from other setup devices 104 and/or intended for other medical devices 102.

The medical device 102 may encode the device data in response to an event, such as when the medical device 102 is powered on, or when a user interface control (e.g., a button or menu option) is activated. To encode the device data (including public key, device identifier, session identifier, other data, or any combination thereof), the medical device 102 may generate a visual encoding such as a one-dimensional bar code, a two-dimensional quick response (“QR”) code, a two-dimensional Aztek barcode, a two-dimensional data matrix, or any other suitable encoding. The medical device 102 may then display encoded device data 110 on a display of the medical device 102, as shown in FIG. 1.

In some embodiments, the medical device 102 may generate a non-visual encoding of the device data. For example, the medical device 102 may generate a digital version of encoded device data that is transmitted to—or read by a sensor of—the setup device 104, such as using dynamic near field communication (“NFC”), the Bluetooth® protocol, the Bluetooth® Low Energy (“BLE”) protocol, the Zigbee® protocol, dynamic radio frequency identification (“RFID”), or another suitable wireless communication technology or protocol.

At [2], the setup device 104 may obtain the encoded device data from the medical device 102. In some embodiments, to obtain the encoded device data, the setup device 104 device scans a component of the medical device 102. For example, if the encoded device data is presented on a graphical display of the medical device 102, a user of the setup device 104 may scan the presentation using an optical sensor such as a camera. Once the setup device 104 has scanned the encoded device data, the setup device 104 can obtain individual data items by decoding the encoded device data. For example, the decoding process may produce a public key of the medical device 102, a device identifier of the medical device 102, a one-time session identifier to be used to establish a communication session with the medical device 102, other data, or any combination thereof. Advantageously, having the setup device 104 scan encoded data off of the medical device 102 as opposed to having the medical device 102 scan connection data off of the setup device 104 relieves a need to have scanning hardware on the medical device 102.

At [3], the setup device 104 can use the device data received from the medical device 102 to generate direct connection data for use in establishing a direct connection that includes a mutually authenticated secure communication session established over a direct wireless connection with the medical device 102. The direct wireless connection is “direct” in the sense that there are no intermediary devices or external network infrastructure involved in the communication path between the setup device 104 and the medical device 102. In some embodiments, a secure communication session may be created as part of a Wi-Fi Direct® group. For example, the setup device 104 creates a Wi-Fi Direct group with a network name and a passphrase. Any device that is to connect to the group will need to know the network name and the passphrase. The setup device 104 may include, in the direct connection data, the network name and passphrase as credentials to be used to join the Wi-Fi Direct group. In some embodiments, the secure communication session may be created using a different protocol or technology, such as Bluetooth, Bluetooth Low Energy (BLE), Zigbee, NFC, or another suitable wireless communication technology or protocol.

The direct connection data may include other information, such as the device identifier and/or session identifier that the medical device 102 can use to confirm that the direct connection data is intended for—and expected by—the medical device 102. To securely provide the direct connection data to the medical device 102, the setup device 104 may encrypt the direct connection data—or a portion thereof, such as the connection credentials—using the public key of the medical device 102. Thus, the direct connection data will only be decryptable using the corresponding secret key of the medical device 102. For future secure communication with the medical device 102, the setup device 104 may add the public key of the medical device 102 to a trust manager or other security subsystem.

At [4], the setup device 104 can broadcast or otherwise share the direct connection data. In some embodiments where broadcasting is supported, the broadcast is made as part of a private group communication, such as a Wi-Fi Direct group session as described above. For example, the setup device 104 may broadcast the encrypted credentials (along with other device data) in the Wi-Fi Direct broadcast channel. The medical device 102 may be configured to listen on the Wi-Fi Direct broadcast channel for such broadcasts. In some embodiments the setup device 104 can send the direct connection data directly to the medical device 102; the authentication in such systems may be ensured through the requirement of close physical proximity (e.g., NFC) or a user entering or confirming PINs displayed on the screens of the setup device 104 and the medical device 102.

At [5], the medical device 102 identifies the direct connection data shared by the setup device 104. The medical device 102 may identify the direct connection data based on the presence of the device identifier and/or session identifier—previously encoded by and presented by the medical device 102—in the direct connection data. The medical device 102 may decrypt the credentials using its private key.

At [6], the medical device 102 uses the decrypted credentials to establish a connection with the setup device 104. For example, the medical device 102 may join the setup device's Wi-Fi Direct group named in the direct connection data, and using the passphrase for the Wi-Fi Direct group included in the direct connection data. In some embodiments, the direct connection includes a mutually authenticated secure connection established over a direct wireless connection (e.g., a Wi-Fi Direct connection, Bluetooth, NFC, etc.). The mutually authenticated secure connection is established using a secure protocol, such as SSL or TLS. To establish a mutually authenticated connection, both entities involved will authenticate to each other. In this case, the mutual authentication is possible as the medical device 102 carries with it a root certificate that can verify the setup device's 104 certificate, and the setup device 104 has the public key of the medical device 102 (through step 3), using which it can verify the medical device's 102 certificate.

In some embodiments, the medical device 102 and setup device 104 may establish a secure connection within the direct communication session. For example, the setup device 104 may generate a key pair and a certificate of its own (or may have been provisioned with a key pair and a certificate. In addition, the setup device 104 has added the public key of the medical device 102 to a trust manager to verify the certificate of the medical device 102 (alternatively, the medical device 102 may generate a second key pair for use in establishing the secure connection within the direct communication session, and may provide the public key from this second key pair to the setup device 104 in the encoded device data). The medical device 102 may have an embedded certificate (e.g., a root certificate embedded in secure storage of the medical device 102 during manufacture) that can be used to verify the certificate of the setup device 104.

The medical device 102 and setup device 104 can use their certificates and key pairs to engage in a secure connection handshake protocol to establish a mutually authenticated connection, such as that used in the SSL or TLS protocols. For example, the setup device 104 can send its certificate (e.g., signed/issued by a certificate authority trusted by the medical device) to the medical device. The medical device 102 can verify the certificate using the embedded root certificate. The medical device 102 can send its certificate (e.g., self-signed using the private key of the medical device 102), which the setup device 104 can verify using the public key of the medical device 102 that the setup device 104 previously obtained as described above.

At [7], once the connection has been established, the setup device 104 may provide configuration data to the medical device 102. In some embodiments, a user of the setup device 104 (e.g., a field service engineer) may enter or otherwise provide the configuration data to the setup device 104.

In some embodiments, the configuration data may be or include information to connect to a network in a clinical environment in which the medical device 102 is deployed (or is to be deployed), a server or network appliance in the clinical environment, or a cloud server remote from the clinical environment. For example, the configuration data may include a wireless network configuration data such as a service set identifier (“SSID”) for an in-clinic Wi-Fi network, a pre-shared key (“PSK”) for the in-clinic Wi Fi network, a certificate, other data, or some combination thereof. As another example, the configuration data may include server information, such as a server address and a certificate to authenticate to the server. In some embodiments, the configuration data may be or include settings to be applied to the medical device other than those relating to network connections. For example, the configuration data may include a passcode to the keep the medical device in diagnostic/service mode, a passcode for screen lock, etc. These passcodes can override default values that may be set by the manufacturer or seller.

In some embodiments, the setup device 104 may be configured to provide different configuration data to different medical devices 102, and the various medical devices 102 may or may not be the same type, make, or model as each other. To determine particular configuration data for a particular medical device 102, the setup device 104 may have a device-configuration map that maps individual medical devices or device types to configuration data. Once a medical device 102 establishes a connection with the setup device 104, the setup device 104 can look up the medical device's device identifier in the device-configuration map and send the medical device 102 its unique or otherwise targeted configuration data.

In some embodiments, as shown in [7′], the setup device 104 may provide a pass-through mode in which configuration data is passed from a server 106 to the medical device 102 through the setup device during the direct communication session, rather than configuration data being stored locally on the setup device 104 prior to establishing the direct communication session. An advantage of such a pass-through mode is that the creating, storage, and maintenance of the configuration data for various devices can be completely offloaded to the server 106 for scenarios in which it is undesirable to have sensitive configuration data stored locally on a mobile setup device 104. Thus, the setup device 104 is not exposed to any sensitive customer settings other configuration data but can still seamlessly configure the medical devices 102.

At [8], the medical device 102 may close the connection to the setup device 104 once the configuration data has been obtained. At [9], the medical device 102 may apply the configuration data received from the setup device 104. For example, the medical device 10 may configure a network interface with the SSID, PSK, and certificate to connect to a clinical network.

At [10], the medical device 102 may establish a network connection using the configuration data obtained from the setup device 104. For example, the medical device 102 may connect to a clinical network and communicate with a server 106, such as an in-clinic server or a remote cloud-based server. In some embodiments, the medical device 102 may obtain software updates, drug libraries, operational instructions and data, etc.

### Example Data Communications

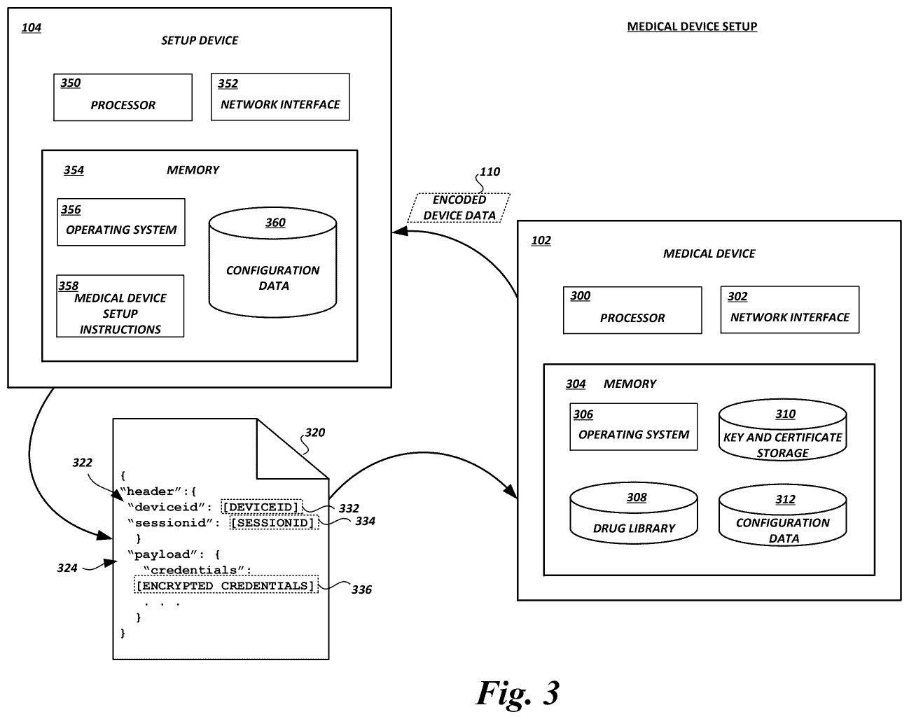

FIG. 3 is a block diagram illustrating exchange of encoded device data and direct connection data between a medical device 102 and a setup device 104 (e.g., at [1]-[5] of FIG. 2), prior to establishment of a direct communication session and the setup device 104 providing configuration data to the medical device 102 (e.g., at [6]-[7] of FIG. 2).

In some embodiments, as shown, the medical device 102 may include: one or more computer processors 300, such as physical central processing units (“CPUs”); one or more network interfaces 302, such as network interface cards (“NICs”); and a computer readable memory 304, such as random access memory (“RAM”) and/or other non-transitory computer-readable media. The computer readable memory 304 may include computer program instructions that the processor 300 executes in order to implement one or more embodiments. For example, the computer readable memory 304 can store an operating system 306 that provides computer program instructions for use by the computer processor 300 in the general administration and operation of the medical device 102. The computer readable memory 304 may also include a drug library 308 with data regarding medications that may be administered using the medical device 102. The medical device 102 may also include a key and certificate storage 310 for storing data used for performing cryptographic operations (e.g., key generation, encryption, decryption, etc.). Although the key and certificate storage 310 is shown in FIG. 3 as being within memory 304, in some embodiments, the key and certificate storage 310 may be physical separated from the memory 304 and from other components of the medical device 102. For example, the key and certificate storage 310 may be stored in a non-volatile secure storage area accessible only by a cryptography subsystem (not shown). The computer readable memory 304 may also include configuration data 312 received from a setup device 104, server 106, or other source as described herein.

In some embodiments, the medical device 102 may be or include an infusion pump with various components to perform infusion pump operations. For example, an infusion pump may include a motor controller unit (“MCU”) configured to control a motor (not shown) that dispenses medication from a medication container based on instructions received via the clinical environment network and/or via a user interface of the medical device 102.

In some embodiments, as shown, the setup device 104 may include: one or more computer processors 350, such as physical CPUs; one or more network interfaces 352, such as NICs; and a computer readable memory 354, such as RAM and/or other non-transitory computer-readable media. The computer readable memory 354 may include computer program instructions that the computer processor 350 executes in order to implement one or more embodiments. For example, the computer readable memory 354 can store an operating system 356 that provides computer program instructions for use by the computer processor 350 in the general administration and operation of the setup device 104. The computer readable memory 354 may also include medical device setup instructions 358 for generating direct connection data and providing configuration data as described herein.

In some embodiments, the computer readable memory 354 may also include configuration data 360 for use in configuring medical devices 102 to access a network of a clinical environment. For example, when a medical device 102 is manufactured, it may be capable of communication over networks (e.g., it may include a network interface and related software), but may not be configured for accessing any specific network. In order to access a specific network when deployed within a clinical environment 100, the medical device 102 may require various configuration settings (e.g., network name, network addresses, etc.). The configuration data 360 may include such configuration settings, or data from which the configuration settings can be derived.

To configure the medical device 102, the setup device 104 may obtain encoded device data 110 that is presented by the medical device 102, as described in greater detail above. Subsequently, the setup device 104 may provide direct connection data 320 to the medical device 102 for use in establishing a direct, secure connection with the setup device 104. In some embodiments, the direct connection data 320 may be provided in a cryptographically signed data token or other data structure that may or may not include other data items. For example, the identity data may be provided in a JavaScript Object Notation (“JSON”) file, a JSON Web Token (“JWT”), an extensible Markup Language (“XML”) file, or some other structured data object.

In some embodiments, as shown, the direct connection data 320 may include various items, such as a header 322 to be identified by the medical device 102, and a payload 324 to be used by the medical device 102 to establish a connection to the setup device 104. For example, the header 322 may include a device identifier 332 and/or a session identifier 334 originally received by the setup device 104 as part of the encoded device data 110. The device identifier 332 may include a unique device ID that is assigned to the medical device 102. For example, the device identifier may be a numeric or alphanumeric string generated using an algorithm that produces unique or substantially unique data (e.g., based on a pseudo-random number generator, a hash function, other functions, or some combination thereof). As another example, the identity data may be selected from a listing of unique identifiers to be assigned to medical devices. The payload 324 may include encrypted credentials 336, such as a network name and passphrase, that the medical device 102 is to use to connect to the setup device 104.

### Time Configuration

In some embodiments, the medical device 102 may include a small battery, such as a super capacitor or “super cap” battery, that powers an internal clock even when the medical device 102 is powered down, unplugged, or has exhausted its primary battery. The internal clock may be used for, among other things, determining whether a certificate is valid (e.g., by comparing the effective date of the certificate to the date of the internal clock). The internal clock may initially be set during manufacturing, and the super cap battery is installed to allow the internal clock to maintain the time, thus allowing an embedded certificate to be considered valid during the setup process described herein. However, after manufacture, the medical device 102 may end up in storage either at the factory or the clinical site. If the medical device 102 is stored for a long period of time, the super cap battery may eventually become exhausted and the medical device 102 can no longer maintain the correct current date/time. For example, the medical device 102 may default to a value such as Jan. 1, 1970. The certificate(s) embedded on medical device 102 to securely connect with either a clinical network or a setup device, or a certificate on a server 106, may have an effective or start time, such as Nov. 1, 2023. When the medical device's internal clock is set back to the older default time, such certificates become unacceptable or unverifiable.

One way to prevent this scenario is to manually set the time on the medical device 102 prior to performing the setup process described herein. However, such manual time configuration can be tedious and costly, particularly when there are dozens, hundreds, or more medical devices 102 to be configured. To address this scenario, the setup device 104 can provide the current time to the medical device 102 prior to—or as part of—the secure configuration process. For example, the setup device 104 gets encoded device data from the medical device 102 and uses the encoded device data to set up a connection to the medical device 102 and securely send a signed current timestamp to the medical device 102. The medical device 102, although it may not have a current time set (other than the default), can verify that the setup device 104 is trusted using the root certificate it has embedded. In some embodiments, once the medical device 102 receives a valid current timestamp from the setup device 104, the medical device 102 applies that time as its own time. After this, the medical device 102 can perform any time-related checks in certificate verification according to established methods. For example, once it has the current timestamp from the setup device 104, the medical device 102 can use specific APIs that also accept the timestamp during certificate validation.

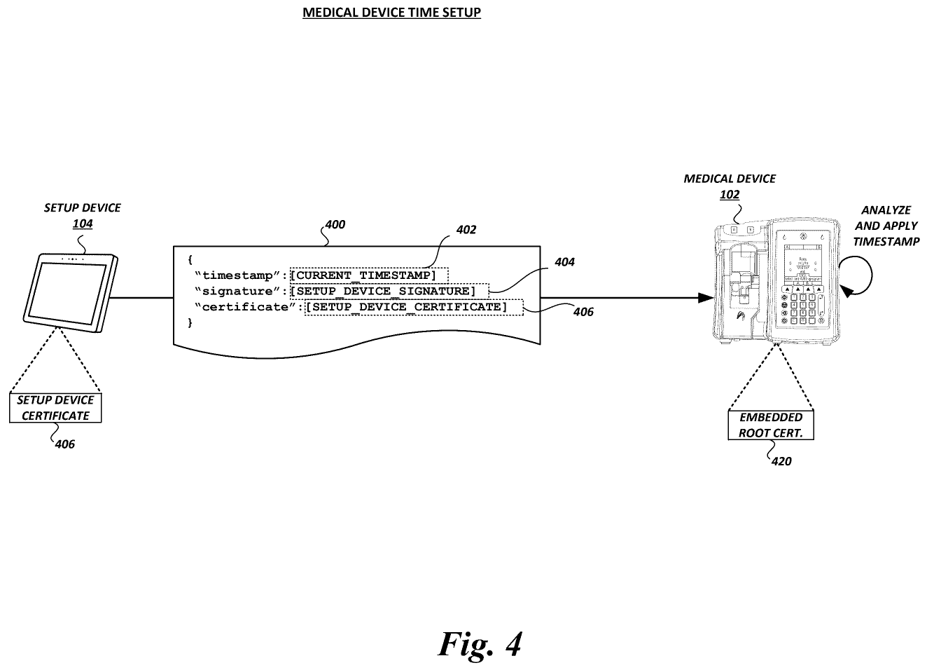

FIG. 4 is a block diagram illustrating a communication of time data 400 from the setup device 104 to the medical device 102. As shown, the medical device 102 has a setup device certificate 406, and the medical device 102 has an embedded root certificate 420 for verifying the setup device 104.

The setup device 104 may provide time data 400 to the medical device 102 prior to, or as part of, the transmission of direct connection data to the medical device 102. In some embodiments, as shown, time data 400 may include a current timestamp 402 determined by an internal clock of the setup device 104, a server in a factory or clinical environment, or some other source. The setup device 104 may also include, in the time data 400, a signature 404 signed using a private key of the setup device 104 and for which the medical device 102 has access to the corresponding public key (e.g., the medical device 102 has an embedded root certificate 420). The setup device 104 may also include, in the time data 400, the setup device certificate 406 for verification of the setup device 104. Upon receipt of time data 400 from the setup device 104, the medical device 102 may process and determine whether to apply the current timestamp included herein.

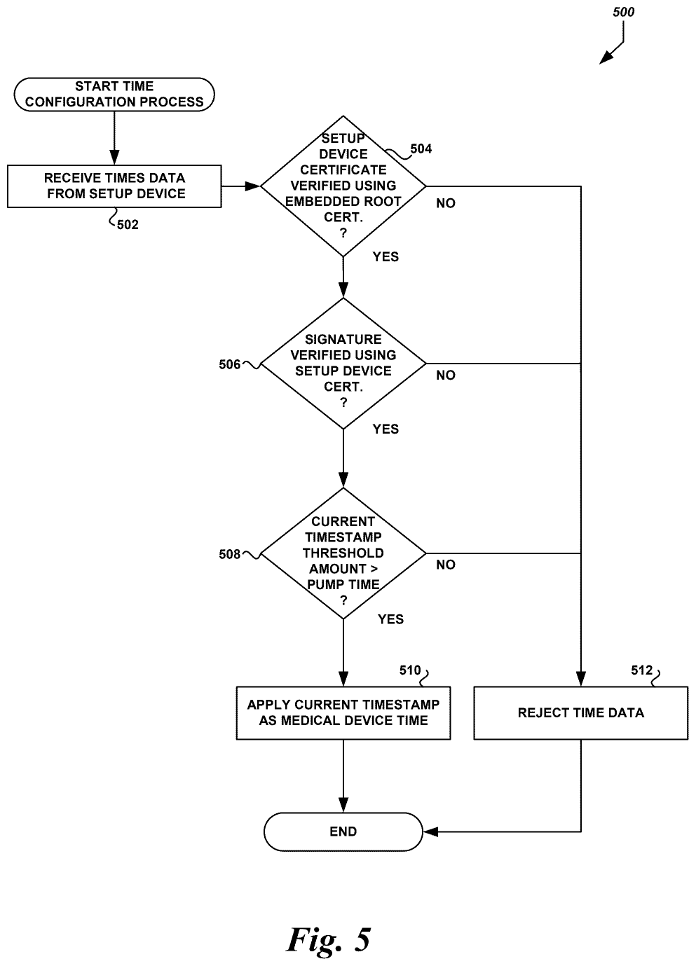

FIG. 5 illustrates a routine 500 that the medical device 102 may perform to verify the source of time data 400 prior to applying a current timestamp 402 included in the time data 400. Routine 500 may begin in response to an event, such as when the medical device 102 is powered on. When the routine 500 is initiated, a set of executable program instructions stored on one or more non-transitory computer-readable media (e.g., hard drive, flash memory, removable media, etc.) may be loaded into memory (e.g., RAM) of the medical device 102 and executed.

At block 502, the medical device 102 can receive time data 400 from a setup device 104. The time data 400 may be received prior to, or as part of, direct connection data that the setup device 104 broadcasts.

At decision block 504, the medical device 102 can determine whether the setup device certificate 406 is able to be verified. The medical device 102 may use the embedded root certificate 420 to verify the certificate 406 in the time data 400. If the setup device certificate 406 is verified, the routine 500 proceeds to decision block 506. Otherwise, if the setup device certificate 406 is not verified, the medical device 102 rejects the time data 400 at block 512.

At decision block 506, the medical device 102 can determine whether the signature 404 is able to be verified. The medical device 102 may use the setup device certificate 406 to verify the signature 404. If the signature 404 is verified, the routine 500 proceeds to decision block 508. Otherwise, if the signature 404 is not verified, the medical device 102 rejects the time data 400 at block 512.

At decision block 508, the medical device 102 can determine whether the current timestamp 402 in the time data 400 is within a threshold amount of time of the current time maintained by the internal clock of the medical device 102. For example, the threshold may be n milliseconds, seconds, minutes, or hours, where n is any predetermined number. If the current timestamp 402 is not within the threshold amount of time of the current time, then the routine 500 may proceed to block 510 where the current timestamp 402 is adopted as the current internal time of the medical device 102. Otherwise, if the current timestamp 402 is within the threshold amount of time of the current time maintained by the internal clock of the medical device 102, the medical device 102 rejects the time data 400 at block 512.

### Terminology and Additional Considerations

All of the methods and tasks described herein may be performed and fully automated by a computer system. The computer system may, in some cases, include multiple distinct computers or computing devices (e.g., physical servers, workstations, storage arrays, cloud computing resources, etc.) that communicate and interoperate over a network to perform the described functions. Each such computing device typically includes a processor (or multiple processors) that executes program instructions or modules stored in a memory or other non-transitory computer-readable storage medium or device (e.g., solid state storage devices, disk drives, etc.). The various functions disclosed herein may be embodied in such program instructions, or may be implemented in application-specific circuitry (e.g., ASICs or FPGAs) of the computer system. Where the computer system includes multiple computing devices, these devices may, but need not, be co-located. The results of the disclosed methods and tasks may be persistently stored by transforming physical storage devices, such as solid-state memory chips or magnetic disks, into a different state. In some embodiments, the computer system may be a cloud-based computing system whose processing resources are shared by multiple distinct business entities or other users.

Depending on the embodiment, certain acts, events, or functions of any of the processes or algorithms described herein can be performed in a different sequence, can be added, merged, or left out altogether (e.g., not all described operations or events are necessary for the practice of the algorithm). Moreover, in certain embodiments, operations or events can be performed concurrently, e.g., through multi-threaded processing, interrupt processing, or multiple processors or processor cores or on other parallel architectures, rather than sequentially.

The various illustrative logical blocks, modules, routines, and algorithm steps described in connection with the embodiments disclosed herein can be implemented as electronic hardware, or combinations of electronic hardware and computer software. To clearly illustrate this interchangeability, various illustrative components, blocks, modules, and steps have been described above generally in terms of their functionality. Whether such functionality is implemented as hardware, or as software that runs on hardware, depends upon the particular application and design constraints imposed on the overall system. The described functionality can be implemented in varying ways for each particular application, but such implementation decisions should not be interpreted as causing a departure from the scope of the disclosure.

Moreover, the various illustrative logical blocks and modules described in connection with the embodiments disclosed herein can be implemented or performed by a machine, such as a processor device, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor device can be a microprocessor, but in the alternative, the processor device can be a controller, microcontroller, or state machine, combinations of the same, or the like. A processor device can include electrical circuitry configured to process computer-executable instructions. In another embodiment, a processor device includes an FPGA or other programmable device that performs logic operations without processing computer-executable instructions. A processor device can also be implemented as a combination of computing devices, e.g., a combination of a DSP and a microprocessor, a plurality of microprocessors, one or more microprocessors in conjunction with a DSP core, or any other such configuration. Although described herein primarily with respect to digital technology, a processor device may also include primarily analog components. For example, some or all of the algorithms described herein may be implemented in analog circuitry or mixed analog and digital circuitry. A computing environment can include any type of computer system, including, but not limited to, a computer system based on a microprocessor, a mainframe computer, a digital signal processor, a portable computing device, a device controller, or a computational engine within an appliance, to name a few.

The elements of a method, process, routine, or algorithm described in connection with the embodiments disclosed herein can be embodied directly in hardware, in a software module executed by a processor device, or in a combination of the two. A software module can reside in RAM memory, flash memory, ROM memory, EPROM memory, EEPROM memory, registers, hard disk, a removable disk, a CD-ROM, or any other form of a non-transitory computer-readable storage medium. An exemplary storage medium can be coupled to the processor device such that the processor device can read information from, and write information to, the storage medium. In the alternative, the storage medium can be integral to the processor device. The processor device and the storage medium can reside in an ASIC. The ASIC can reside in a user terminal. In the alternative, the processor device and the storage medium can reside as discrete components in a user terminal.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without other input or prompting, whether these features, elements and/or steps are included or are to be performed in any particular embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y, Z,” unless specifically stated otherwise, is otherwise understood with the context as used in general to present that an item, term, etc., may be either X, Y, or Z, or any combination thereof (e.g., X, Y, and/or Z). Thus, such disjunctive language is not generally intended to, and should not, imply that certain embodiments require at least one of X, at least one of Y, or at least one of Z to each be present.

Unless otherwise explicitly stated, articles such as “a” or “an” should generally be interpreted to include one or more described items. Accordingly, phrases such as “a device configured to” are intended to include one or more recited devices. Such one or more recited devices can also be collectively configured to carry out the stated recitations. For example, “a processor configured to carry out recitations A, B and C” can include a first processor configured to carry out recitation A working in conjunction with a second processor configured to carry out recitations B and C.

While the above detailed description has shown, described, and pointed out novel features as applied to various embodiments, it can be understood that various omissions, substitutions, and changes in the form and details of the devices or algorithms illustrated can be made without departing from the spirit of the disclosure. As can be recognized, certain embodiments described herein can be embodied within a form that does not provide all of the features and benefits set forth herein, as some features can be used or practiced separately from others. The scope of certain embodiments disclosed herein is indicated by the appended claims rather than by the foregoing description. All changes which come within the meaning and range of equivalency of the claims are to be embraced within their scope.

1. A system for secure configuration of medical devices, the system comprising:
a medical device comprising one or more processors, a secure memory, and a first wireless network interface; and
a setup device comprising one or more processors and a second wireless network interface;
wherein the medical device is configured to:
present encoded device data representing a public key of the medical device, a device identifier of the medical device, and a session identifier of a direct communication session to be established with the setup device;
receive, from the setup device, direct connection data including credentials, encrypted using the public key, for establishing the direct communication session;
decrypt the credentials using a private key corresponding to the public key;
establish the direct communication session using the credentials; and
receive, from the setup device via the direct communication session, configuration data for communicating with a server over a clinical network; and
wherein the setup device is configured to:
scan the encoded device data presented by the medical device;
generate the direct connection data, including the credentials, encrypted using the public key;
broadcast the direct connection data;
establish the direct communication session with the medical device; and
send the configuration data to the medical device.

2. The system of claim 1, wherein the medical device is an infusion pump.

3. (canceled)

4. (canceled)

5. (canceled)

6. The system of claim 1, wherein the setup device is further configured to establish a secure connection with the medical device using a certificate and a keypair of the setup device for authenticating the setup device to the medical device and using the public key of the medical device to authenticate the medical device.

7. The system of claim 1, wherein the setup device is further configured to send time data to the medical device, wherein the time data comprises a current timestamp.

8. The system of claim 7, wherein the medical device is further configured to:
receive the time data from the setup device;
determine that the time data is not within a threshold period of time of a current time of the medical device as maintained by an internal clock of the medical device; and
determine to apply the current timestamp from the time data as the current time of the medical device.

9. The system of claim 7, wherein the medical device is further configured to:
receive the time data from the setup device;
determine that the time data is within a threshold period of time of a current time of the medical device as maintained by an internal clock of the medical device; and
determine to not apply the current timestamp from the time data as the current time of the medical device.

10. The system of claim 1, wherein the medical device comprises a display screen, and where the medical device is further configured to present the encoded device data on the display screen, wherein the encoded device data comprises one of a bar code or a QR code.

11. (canceled)

12. The system of claim 10, wherein the setup device comprises an optical scanner, and wherein the setup device is configured to scan the encoded device data presented on the display screen of the medical device.

13. The system of claim 1, wherein the medical device is further configured to determine that the device identifier and the session identifier are present in the direct connection data, and wherein the medical device decrypts the credentials in response to determining that the device identifier and the session identifier are present in the direct connection data.

14. The system of claim 1, wherein the medical device is further configured to discard the private key and the public key in response to receiving the configuration data from the setup device via the direct communication session.

15. (canceled)

16. The system of claim 1, wherein the medical device is further configured to use a second private key for certificate-based authentication of the medical device during a handshake protocol for establishing the direct communication session with the setup device.

17. (canceled)

18. The system of claim 1, wherein the medical device is further configured to use the private key for certificate-based authentication of the medical device during a handshake protocol for establishing the direct communication session with the setup device.

19. (canceled)

20. (canceled)

21. (canceled)

22. A computer-implemented method for secure configuration of medical devices, comprising:
as performed by a medical device comprising one or more processors, a secure memory, and a first wireless network interface,
presenting encoded device data representing a public key of the medical device, a device identifier of the medical device, and a session identifier of a direct communication session to be established with a setup device;
receiving, from the setup device, direct connection data including credentials, encrypted using the public key, for establishing the direct communication session;
decrypting the credentials using a private key corresponding to the public key;
establishing the direct communication session using the credentials; and
receiving, from the setup device via the direct communication session, configuration data for communicating with a server over a clinical network.

23. The computer-implemented method of claim 22, further comprising:
receiving time data from the setup device, wherein the time data comprises a current timestamp;
determining that the time data is not within a threshold period of time of a current time of the medical device as maintained by an internal clock of the medical device; and
determining to apply the current timestamp from the time data as the current time of the medical device.

24. The computer-implemented method of claim 22, further comprising:
receiving time data from the setup device, wherein the time data comprises a current timestamp;
determining that the time data is within a threshold period of time of a current time of the medical device as maintained by an internal clock of the medical device; and
determining to not apply the current timestamp from the time data as the current time of the medical device.

25. The computer-implemented method of claim 22, further comprising presenting the encoded device data on a display screen of the medical device, wherein presenting the encoded device data comprises presenting one of a one-dimensional bar code, a two-dimensional quick response (OR) code, a two-dimensional Aztek barcode, or a two-dimensional data matrix.

26. (canceled)

27. An infusion pump comprising one or more processors, a secure memory, and a first wireless network interface, wherein the infusion pump is configured to:
present encoded device data representing a public key of the infusion pump, a device identifier of the infusion pump, and a session identifier of a direct communication session to be established with a setup device;
receive, from the setup device, direct connection data including credentials, encrypted using the public key, for establishing the direct communication session;
decrypt the credentials using a private key corresponding to the public key;
establish the direct communication session using the credentials; and
receive, from the setup device via the direct communication session, configuration data for communicating with a server over a clinical network.

28. The infusion pump of claim 27, further configured to:
receive time data from the setup device, wherein the time data comprises a current timestamp;
determine that the time data is not within a threshold period of time of a current time maintained by an internal clock of the infusion pump; and
determine to apply the current timestamp from the time data as the current time of the infusion pump.

29. The infusion pump of claim 27, further configured to:
receive time data from the setup device, wherein the time data comprises a current timestamp;
determine that the time data is within a threshold period of time of a current time maintained by an internal clock of the infusion pump; and
determine to not apply the current timestamp from the time data as the current time of the infusion pump.

30. The infusion pump of claim 27, further configured to present the encoded device data on a display screen of the infusion pump, wherein the encoded device data comprises one of a one-dimensional bar code, a two-dimensional quick response (OR) code, a two-dimensional Aztek barcode, or a two-dimensional data matrix.

31. (canceled)

32. (canceled)

33. (canceled)

34. (canceled)

35. (canceled)

36. (canceled)
